(19) United States
(12) Patent Application Publication
(10) Pub. No.: US 2016/0220507 A1
(43) Pub. Date: Aug. 4, 2016

(54) KIT FOR SUSTAINED TRANSDERMAL DRUG DELIVERY USING LIQUID OR SEMISOLID FORMULATIONS AND METHOD OF USING THE SAME

(71) Applicant: Jie Zhang, Salt Lake City, UT (US)

(72) Inventor: Jie Zhang, Salt Lake City, UT (US)

(21) Appl. No.: 14/903,979

(22) PCT Filed: Jul. 10, 2014
(86) PCT No.: PCT/US14/46080

§ 371 Date: Jan. 8, 2016

Related U.S. Application Data
(60) Provisional application No. 61/844,515, filed on Jul. 10, 2013.

(51) Int. Cl. A61K 9/70; A61K 31/167; A61K 31/196; A61K 9/06

(52) U.S. Cl. CPC A61K 9/7084; A61K 9/06; A61K 31/167; A61K 31/196

(57) ABSTRACT

A kit for sustained transdermal drug delivery using liquid or semisolid drug formulations and methods of using the kit are disclosed. The kit may comprise a mechanism for delivering a drug across skin for a sustained period of time using a liquid or semisolid drug formulation that, without the structured sheet of the kit, is not suitable for sustained drug delivery. In some embodiments, the methods may be used for treating joint or muscle pain.

### TECHNICAL FIELD

The present disclosure is directed to kits for sustained transdermal drug delivery using liquid or semisolid formulations and methods of using such kits.

### BRIEF DESCRIPTION OF THE DRAWINGS

The embodiments disclosed herein will become more fully apparent from the following description and appended claims, taken in conjunction with the accompanying drawings. These drawings depict only typical embodiments, which will be described with additional specificity and detail through use of the accompanying drawings in which:

FIG. 1 is a perspective view of an embodiment of a kit for topical delivery of a drug formulation.

FIG. 2 depicts a method of using the embodiment of FIG. 1.

FIG. 3 is an exploded view of a portion of the embodiment of FIG. 1.

FIG. 4 is a perspective view of an embodiment of the current disclosure secured to a target skin area.

FIG. 5A is a cross-section of the embodiment of FIG. 4 taken through line 5A-5A.

FIG. 5B is a cross-section of another embodiment taken through line 5B-5B.

### BACKGROUND

Liquid and semisolid drug formulations, such as solutions and gels, are widely used to treat various skin conditions and open wounds, but they are usually not suitable for sustained transdermal drug delivery (continuous transdermal drug absorption for many hours or days) because of the difficulty of keeping a liquid or semisolid drug formulation on the skin for an extended period. Such formulations, when applied on the skin, can be easily removed by contact with external objects including, but not limited to, clothing and furniture. Additionally, some ingredients of liquid and solid drug formulations, such as solvents, are volatile and quickly evaporate after application of the formulation. Solvent evaporation can significantly reduce or even stop absorption of a drug into the skin. As a result, a user often has to apply a liquid or semisolid drug formulation multiple times a day to deliver the desired amount of the drug across the skin. For example, PENNSAID®, a topical diclofenac solution, must be applied to the skin of the knee four times a day.

Patches, including matrix and reservoir types, are often used to achieve sustained transdermal drug delivery because the typical patch is capable of adhering to the skin for extended periods and the drug formulation in the patch is protected from contact with external objects. Patches, however, require the drug formulation to be in a solid state or contained in a solid bag. In matrix patches, the drug is contained in a solid state formulation. In reservoir patches, the drug is usually in a semisolid state formulation, such as gel, which is contained in a flat bag. One side of the bag usually comprises a drug-permeable membrane coated with an adhesive. When the adhesive is applied to the skin, the patch adheres to the skin, and the drug must permeate through both the membrane and the adhesive to reach the skin.

In some situations, however, drugs intended for sustained transdermal delivery are ideally formulated in a liquid or semisolid formulation and thus cannot be used in a matrix patch. Additionally, separating liquid and semisolid formulations from the skin by a membrane and an adhesive, as in a reservoir patch, can be problematic. In these situations, it is undesirable or impossible to put the formulation in a matrix or reservoir patch or to apply the formulation directly to the skin.

Consequently, there is an unmet need for an apparatus and/or method for maintaining liquid and/or semisolid drug formulations, which may include volatile solvents, on the skin for extended periods.

### DETAILED DESCRIPTION

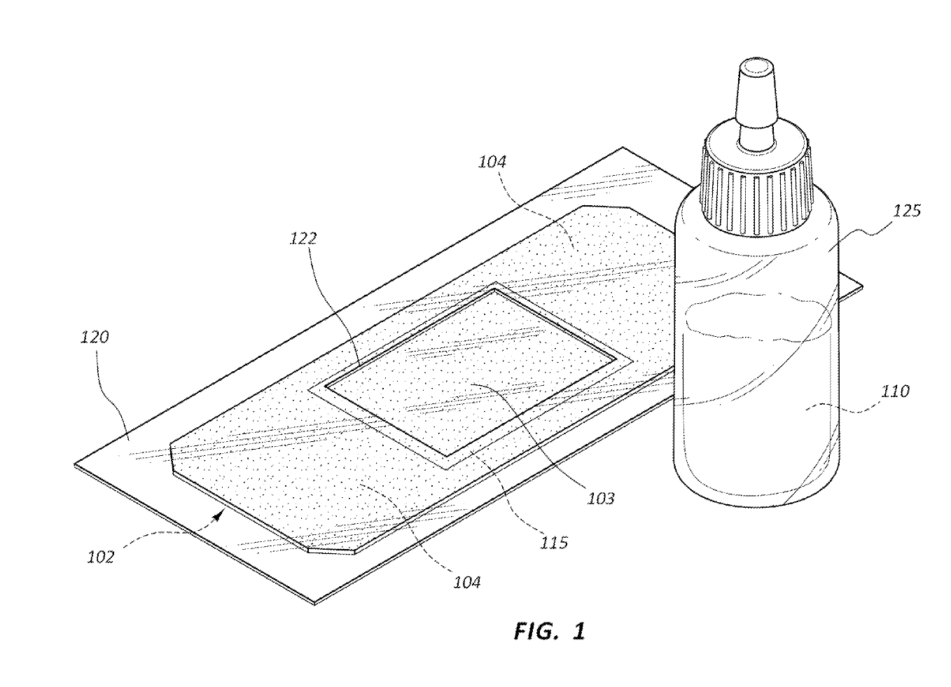

FIG. 1 is a perspective view of an embodiment of a kit for topical delivery of a drug formulation. As illustrated, a kit of the present disclosure may comprise two components: (1) a liquid or semisolid formulation 110 comprising a drug; and (2) a structured sheet 102 comprising a drug retention area 103 for retaining the drug formulation 110 on the skin for extended periods and an adhesive elastic area 104 for adhering or securing the structured sheet 102 to the skin during drug application.

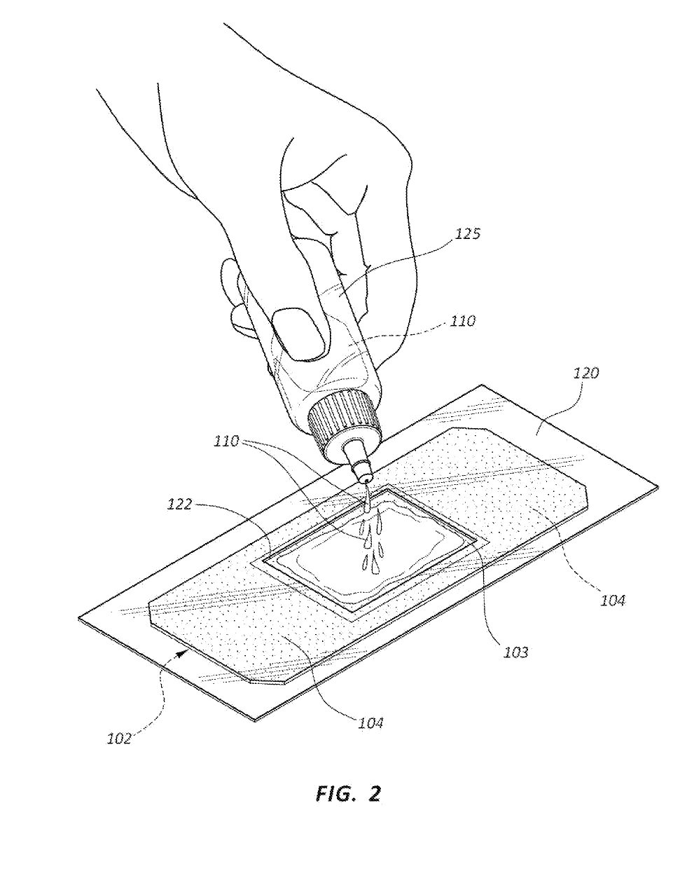

To use the kit, the drug formulation 110 may be applied, spread, sprayed, and/or dripped on the skin. The structured sheet 102 may then be applied to the skin in such a way that the drug formulation 110 on the skin area is covered by the drug retention area 103 of the structured sheet 102, and the adhesive elastic area 104 of the structured sheet 102 is placed on skin areas not covered by the drug formulation 110. Such a configuration may facilitate affixing the structured sheet 102 to the skin. Alternatively, as depicted in FIG. 2, the drug formulation 110 can be applied onto the drug retention area 103 of the structured sheet 102 before the structured sheet 102 is applied to the skin. After the drug formulation 110 and the structured sheet 102 are applied using the aforementioned techniques, the drug formulation 110 may thus be covered by the drug retention area 103 of the structured sheet 102, and may be at least partially protected from evaporation and touching by external objects. In this way, the drug formulation 110 can stay on the skin area for an extended period (for example, many hours or many days). Such an extended period may be required for sustained drug delivery.

The terms “moisture vapor transfer rate” and “MVTR” refer to the moisture vapor transfer rate across a film or tape, or a layer of another kind of material, as measured by standard methods used in the medical tape and film industry in the United States (U.S.), such as that used by the 3M Company, and known by one skilled in the art. “MVTR higher than 5000 g/m²/24 hour” indicates that a material's MVTR is that high as measured by standard methods or that the material (e.g., fabric cloth) may be so porous that liquid water can pass through it.

The term “barrier film” refers to a layer of material, such as a plastic film or tape, which is a barrier to bulk liquid. A barrier film can have a MVTR lower than 2000 g/m²/24 hour, or lower than 800 g/m²/24 hour, or even lower than 100 g/m²/24 hour. Many films or tapes, such as polyethylene film or tape and polyurethane film or tape, can be used as a barrier film.

The term “structured sheet” refers to a sheet with two distinctive areas: (1) a drug retention area for retaining a drug formulation on the skin for extended periods; and (2) an adhesive elastic area for adhering the structured sheet to the skin during drug formulation application.

The term “breathable” refers to a material with a certain high MVTR, for example, a MVTR higher than about 800 g/m²/24 hour, higher than about 1200 g/m²/24 hour, or higher than about 5000 g/m²/24 hour.

The term “elastic”, when used to describe the drug retention area or the adhesive elastic area of the structured sheet, refers to that part of the structured sheet that can be stretched by a stretching force to increase its original length by at least 5%, or by at least 10%, in at least one direction, and that is able to recover its original length when the stretching force is removed.

The terms “drug” or “drugs” refer to any agent for treating musculoskeletal pain or other medical conditions that may be treated by drug delivery into regional tissues under the skin or the systemic circulation. In certain embodiments, the terms “drug” or “drugs” do not include any agents such as antibiotics for treating wounded skin (e.g., skin with a damaged stratum corneum layer) or open wounds, but do include local anesthetic and anti-inflammatory drugs. Therefore, drugs “not for treating a wound or skin infection” include anti-inflammatory and local anesthetic drugs.

The term “liquid or semisolid formulation” refers to formulations that are low viscosity liquids and viscous liquids, including solutions, gels, creams, ointments, oil-in-water emulsions, water-in-oil emulsions, solid-in-water, or other liquid suspensions.

The terms “target human skin area” and “target skin area” and the like refer to a skin area of a human patient onto which a drug formulation and a structured sheet of the current disclosure is applied to treat a medical condition.

The term “approved drug product” refers to a drug or drug product that is approved by U.S. FDA for marketing in the U.S. or approved by a comparable entity for marketing in another country or jurisdiction.

The terms “over-the-counter product” or “OTC product” refer to a drug or drug product that qualifies as an over-the-counter product in the U.S. or as a comparable product in another country or jurisdiction.

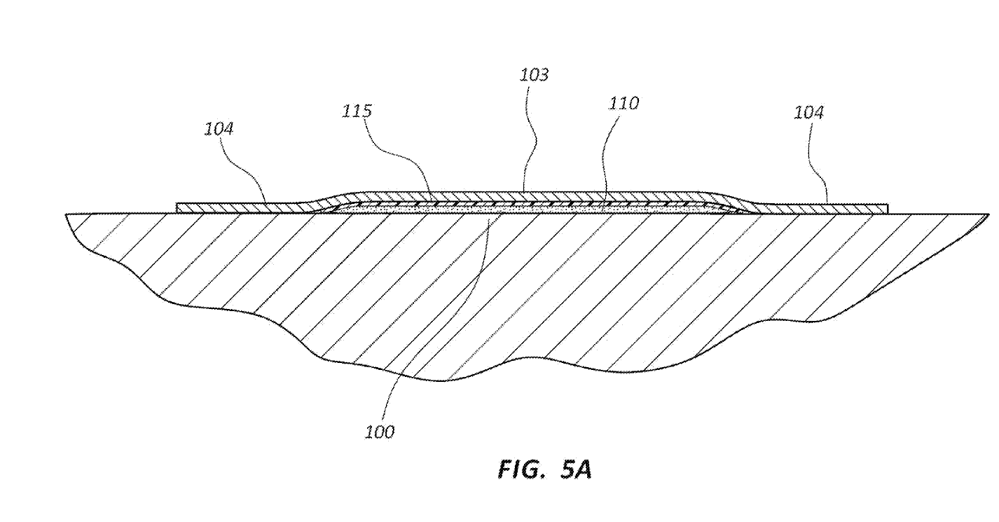

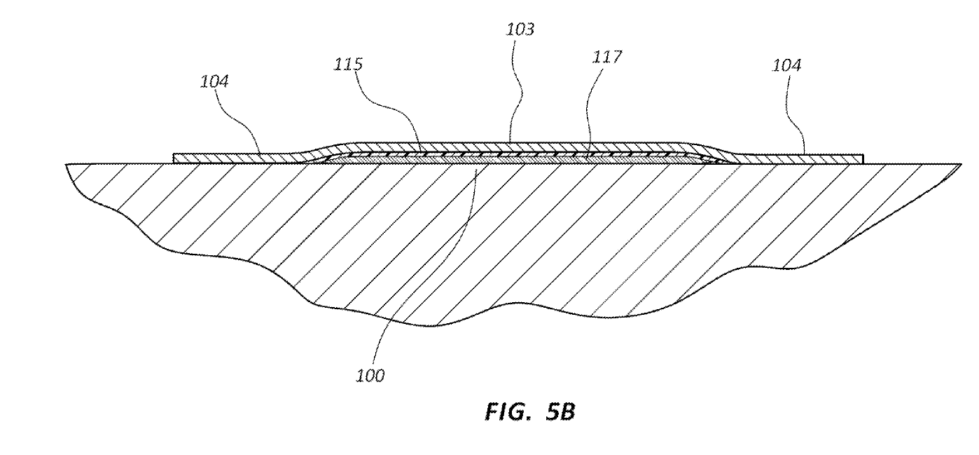

Referring again to FIGS. 1 and 2, in some embodiments, the drug retention area 103 may have a low MVTR, for example, lower than 800 g/m²/24 hour, lower than 400 g/m²/24 hour, lower than 200 g/m²/24 hour, or even lower than 50 g/m²/24 hour, such that the volatile component or components of the liquid drug formulation 110, such as water, may be retained on the skin for an extended period. The drug retention area 103 may also comprise a layer of absorbent material for keeping low viscosity drug formulations in place by absorbing the formulation into the absorbent layer, thus allowing the formulation to contact the skin without flowing away (see, e.g., FIG. 5B).

Optionally, the drug retention area 103 may be elastic, meaning it can be stretched and recover its original shape when the skin is stretched and/or relaxed during a body movement or movements. While the drug retention area 103 can comprise any shape or size, it may be large enough to deliver sufficient amount of a drug across the skin to achieve a desired clinical effect. In some embodiments, the drug retention area 103 may be larger than about 10 cm², larger than about 50 cm², or larger than about 100 cm².

In an embodiment, the adhesive elastic area 104 of the structured sheet 102 may be adhesive to human skin, elastic, and/or breathable so that it may secure the structured sheet 102 to the skin for an extended period, such as many hours or even days, and tolerate sweating. In other embodiments, the adhesive elastic area 104 may be elastic so that it can effectively secure the structured sheet 102 to skin surfaces that are often stretched during movements, such as the skin areas over joints and muscles.

In the structured sheet 102 of some embodiments, the MVTR of the drug retention area 103 can be much lower than that of the adhesive elastic area 104. For example, the MVTR of the drug retention area 103 may be lower than one-fifth, one-tenth, or even one-twentieth of the MVTR of the adhesive elastic area 104.

In one embodiment, the MVTR of the drug retention area 103 of the structured sheet 102 may be lower than about 100 g/m²/24 hour (for example, due to a polyethylene barrier film), while the MVTR of the adhesive elastic area 104 of the structured sheet 102 may be so high that liquid water can permeate through it. In some embodiments, the adhesive elastic area 104 of the structured sheet 102 may comprise a non-woven elastic fabric material coated with an adhesive.

In another embodiment, referring again to FIG. 1, the MVTR of the drug retention area 103 of the structured sheet 102 may be lower than about 100 g/m²/24 hour (for example, due to a polyethylene barrier film 115) while the MVTR of the adhesive elastic area 104 of the structured sheet 102 may be higher than about 800 g/m²/24 hour. In some embodiments, the adhesive elastic area 104 of the structured sheet 102 may comprise a thin polyurethane film coated with an adhesive.

In yet another embodiment, the MVTR of the drug retention area 103 of the structured sheet 102 may be about 400 g/m²/24 hour (for example, due to a relatively thick polyurethane film) while the MVTR of the adhesive elastic area 104 of the structured sheet 102 may be so high that liquid water can permeate through it.

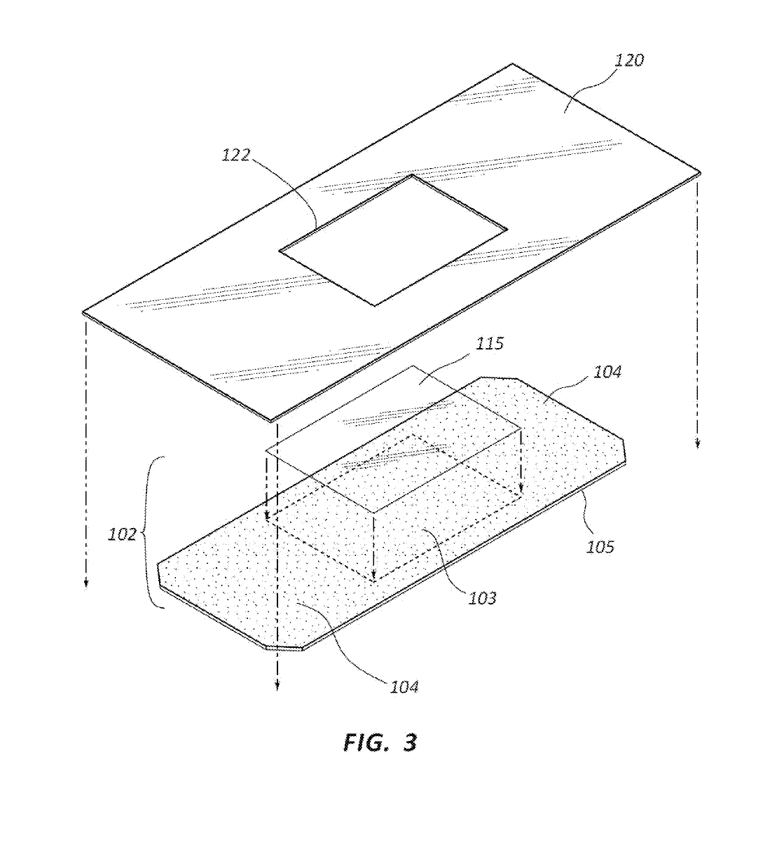

FIG. 3 is an exploded view of the embodiment of the structured sheet 102 of FIG. 1 and a release liner 120. The structured sheet 102, as illustrated, comprises a sheet portion 105 and the barrier film 115. The MVTR of the drug retention area 103, illustrated as the area within the dashed lines, may be achieved, at least in part, by the use of the barrier film 115. For example, a structured sheet 102 can be formed by attaching a piece of the barrier film 115 (for example, polyethylene film with a certain thickness) to a sheet portion 105, comprising an elastic non-woven fabric tape. Such a tape may be very porous with a high MVTR. The area of the tape covered by the barrier film 115 may have a low MVTR and may comprise the drug retention area 103, while the area of the tape not covered by the barrier film 115 may be the adhesive elastic area 104, illustrated as the area outside of the dashed lines, of the structured sheet 102. However, in other embodiments, barrier films (e.g., polyethylene film) may not be very elastic, thus the drug retention area of such a structured sheet may not be very elastic. To overcome this, in some embodiments, the barrier film may be placed on the fabric tape while the tape is stretched, so when the tape shrinks to its original shape, the barrier film may have to wrinkle in order to stay attached to the tape. The drug retention area of such a structured sheet, a structured sheet with wrinkled drug retention area, may be elastic. Therefore, in some embodiments, the drug retention area may comprise a wrinkled barrier film.

In some embodiments, the drug retention area 103 of the structured sheet 102 may be surrounded by the adhesive elastic area 104 in all directions. When such a structured sheet 102 is applied to the skin, with the drug formulation already applied on the drug retention area 103, the drug formulation may be completely isolated from the external environment. This may help keep the drug formulation in the drug retention area 103 and protect the drug formulation from the external environment and/or from solvent evaporation.

As depicted in FIGS. 1 and 3, in certain embodiments the structured sheet 102 can be joined with the release liner 120 comprising a window 122 (e.g., an empty space). The window 122 may facilitate the application of the drug formulation 110 to the drug retention area 103. For example, the window 122 may minimize spreading of the drug formulation 110 into the adhesive elastic area 104, and the release liner 120 may protect the adhesive elastic area 104 during storage and handling.

In some embodiments, the window 122 may be of the same size and shape as the drug retention area 103 in the structured sheet 102, such that when the release liner 120 is joined with the structured sheet 102, the window 122 may coincide with the drug retention area 103 of the structured sheet 102. In this way, the drug retention area 103 may not be covered by the release liner 120, but the adhesive elastic area 104 may be covered by the release liner 120. In some embodiments, when the release liner 120 and the structured sheet 102 are joined, 100% of the drug retention area 103 may not have to coincide with the window 122 of the release liner 120, but at least some portion of the drug retention area 103, including at least about 60% or at least about 80% of the drug retention area 103, may coincide with the window 122. Additionally, while not all of the adhesive elastic area 104 of the structured sheet 102 may be covered by the non-window part of the release liner 120, it may be desirable that most of the adhesive elastic area 104, including at least about 80% of the adhesive elastic area 104, is covered by the non-window part of the release liner 120.

Referring again to FIG. 2, in some embodiments, when using a structured sheet 102 at least partially covered with a release liner 120 comprising a window 122, the user may apply, by spreading, spraying, and/or dripping, the drug formulation 110 directly onto the drug retention area 103 of the structured sheet 102 through the window 122. In this way, if the drug formulation 110 is inadvertently applied to an area larger or beyond the drug retention area 103, the window 122 may limit the drug formulation 110 to the part of the structured sheet 102 that coincides with the window 122, such that the size and/or shape of the transdermal drug delivery area may always be as pre-designed, and the adhesive elastic area 104 may be protected from the drug formulation 110.

In most cases in which patients suffering from a medical condition or medical conditions that may be beneficially treated with drug delivery into the regional tissues or the systemic circulation, the target skin area comprises intact skin. Thus, formulations with good drug permeability across intact skin should be used, and sustained drug delivery may be required. In contrast, skin with a broken or otherwise compromised stratum corneum layer or other layers may comprise abnormal and/or lower barrier properties. Applying a drug formulation of the present disclosure to non-intact skin may cause unpredictable and/or higher than desired absorption of the drug into the body. Thus, application of a drug formulation of the present disclosure to non-intact skin may be undesirable. Therefore, in some embodiments, the formulation or formulations of the present disclosure may only be applied to intact skin.

In certain embodiments, drugs of the present disclosure include, but are not limited to, local anesthetic agents such as lidocaine, tetracaine, prilocaine, and anti-inflammatory agents such as diclofenac and ketoprofen. Drugs that may be used with the present disclosure may also comprise agents for systemic delivery, including hormones such as testosterone and pain medicines such as fentanyl. The drugs that may be used with embodiments of the present disclosure may be in either ionized or unionized form. For a basic drug, the drug may be in the form of free base or a salt (e.g., lidocaine hydrochloride). For an acidic drug, the drug may be in the form of free acid or a salt (e.g., diclofenac sodium).

In certain embodiments, it may be important that the structured sheet and the drug formulation of the current disclosure are correctly used together. For that reason, and for the convenience to the user, it may be desirable to place the structured sheet and the formulation into a container including, but not limited to, a bag or box, and optionally include a use instruction in the same container.

In some embodiments, when a kit of the current disclosure is used to deliver a systemic drug, the skin area in contact with the drug formulation (hence the skin area available for drug delivery) may be defined by the drug retention area of the structured sheet. If some of the drug formulation is applied outside of the drug retention area and into the adhesive elastic area, the volatile component (usually comprising a solvent that may be necessary for drug delivery) may be quickly lost by evaporation through the breathable adhesive elastic area, resulting in the stoppage or significant slowdown of drug delivery to an area of skin outside of the drug retention area. As a result, only the skin area under the drug retention area may be substantially involved in sustained drug delivery. This precision may be similar to drug delivery using a patch, but a patch may only use drug formulations in a solid state or a patch may have to separate the drug formulation from the skin with a membrane and an adhesive layer as discussed above.

As compared to a patch, cost per application for the kits of the current disclosure may be lower or much lower, because one container 125 (e.g., a squeeze bottle), of the present disclosure may contain enough drug formulation for many applications (e.g., tens, or even hundreds, of applications), as compared to a patch's single application (see FIGS. 1 and 2).

Liquid or semisolid formulations, as defined above, may be vulnerable to evaporation of volatile components and unintentional removal by contact by external objects, if applied to the skin without protection. Therefore, in the embodiments of the current disclosure, liquid or semisolid formulations may especially benefit from the structured sheet of the present disclosure and may be retained on the skin for extended periods for sustained transdermal drug delivery.

One advantage of a kit of the present disclosure may be the ability to retain liquid and semisolid formulations on the skin for extended periods such that sustained drug delivery can be achieved. Without the structured sheet, these formulations may not be able to perform sustained drug delivery because they may quickly lose their volatile solvent or solvents via evaporation and they may be removed by touching external objects. This advantage may allow one to develop formulations that are optimized for drug delivery without having to sacrifice the drug delivery ability for obtaining some must have properties such as skin adhesion and/or drug-adhesive compatibility.

Table 1 demonstrates the above-described advantage of certain embodiments of the present disclosure (e.g., Example 1) in comparison with a lidocaine transdermal patch, LIDODERM®. The LIDODERM® patch is used for treating post herpetic neuralgia and various kinds of musculoskeletal pain. In 2012, U.S. annual sales of the LIDODERM® patch were about $1 billion.

TABLE 1

| | A formulation | |
|---|---|---|
| | of the current | |
| | disclosure | |
| | (Example 1, | |
| | see below) | LIDODERM ® Patch |
| Lidocaine concentration | 2.5% | 5% |
| Time needed to | About | More than 5 hours for some |
| anesthetize intact human | 90 minutes | patients. For most patients, |
| skin | | skin anesthesia is never |
| | | achieved in the entire 12 hour |
| | | application time. |

As shown in Table 1, even with twice the concentration of lidocaine (5% vs. 2.5%), LIDODERM® has a much weaker ability to deliver lidocaine across the skin. How quickly a lidocaine formulation can numb the skin is a measure of transdermal lidocaine delivery rates, even when the purpose may not be skin anesthesia. The weak ability of the LIDODERM® patch to deliver lidocaine across the skin is due to the fact that the formulation used in the LIDODERM® patch, a hydrogel, has to be sufficiently adhesive to attach itself to the skin. Being adhesive requires the pH of the formulation to be low; however, the transdermal permeability of lower pH lidocaine formulations is much lower than that of higher pH lidocaine formulations. Therefore, the LIDODERM® patch sacrifices an important efficacy property, transdermal permeability, in order to achieve another necessary property, skin adherence.

Another advantage of the current disclosure is that certain liquid or semisolid drug formulations that are already approved by government authorities for marketing, but that in their current form can only deliver the drug for short periods of time, can now be used for sustained delivery. For example, PENNSAID® diclofenac solution (1.5%) is approved by FDA and is sold on the U.S. market, but has to be used four times a day. Using diclofenac as the formulation component of a kit of the current disclosure, one can achieve sustained delivery of diclofenac, so that once a day application is possible, without having to develop a new formulation. In another example, the drug formulation component of the kit is a 4% lidocaine gel which alone qualifies as an over-the-counter drug product in the U.S. (for applications on skin with certain damage such as insect bites and minor burns) but now can be used for sustained delivery of lidocaine for treating musculoskeletal pain. Therefore, in certain embodiments of the kit of the current disclosure, the drug formulation alone is the same as or would qualify as a generic version of a product already approved by FDA for marketing in the U.S. In other embodiments, the drug formulation in a kit of the current disclosure qualifies or would qualify as an over-the-counter drug product in the U.S. “Would qualify” means the product is expected, by those skilled in the art, to meet the qualification after certain work required by the government authorities (e.g., clinical trials, bioequivalence studies, or certain manufacturing and documentation work) has been performed. “Would qualify” as a generic version of an approved product or as an over-the-counter product includes the situation in which the product of the current disclosure is used for treating medical condition(s) that are different from the medical condition(s) that said approved or over-the-counter product is approved for, and/or in which the method of using the product of the current invention is different from that of said approved or over-the-counter product. For example, the 4% lidocaine formulation in Example 4 “would qualify” as an over-the-counter product in the U.S.A., although a 4% lidocaine gel qualifies as an over-the counter product if used for conditions such as minor burns and insect bites but may not be considered as an over-the-counter product if used for treating joint pain (which can be the purpose of the kit in Example 4). In another example, the PENNSAID® diclofenac solution can be used as the formulation in the kit in Example 3 and can be used once a day, while the original PENNSAID® diclofenac solution is used 4 times a day. This difference in method of use would likely prevent the product from being approved as a generic version of the original product in the U.S.A., but according to the definition of the current disclosure, the PENNSAID® diclofenac solution in Example 3 “would qualify” as the generic product to the original PENNSAID® diclofenac solution. “Would qualify” as a generic version of an approved product or as an over-the-counter product also includes the situations in which the active drug concentration(s) in the formulation of the current invention is about the same as that in the approved product or the over-the-counter product.

Due to the advantages offered by the structured sheet, the sustained drug delivery formulations in the kit of some embodiments of the current disclosure may not need drug concentrations higher than that used in short drug duration products. In some embodiments, the drug formulation contains less than 5% lidocaine, less than 4% lidocaine, or even less than 2% lidocaine. In some embodiments, most of the lidocaine in the formulation is in the base (unionized) form. In some other embodiments, the drug formulation contains less than 3% diclofenac, less than 2% diclofenac, or even less than 1.5% diclofenac.

One embodiment of the current disclosure comprises a kit for delivering a drug across human skin. This kit comprises a liquid or semisolid formulation comprising a drug, and a structured sheet comprising a drug retention area and an adhesive elastic area. In some embodiments, the drug may be either lidocaine at a concentration of 5% or lower or diclofenac at a concentration of 3% or lower. Other drugs are also within the scope of this disclosure. The MVTR of the retention area is lower than about 200 g/m²/24 hour. The MVTR of the adhesive elastic area is higher than 5,000 gram/m²/24 hour.

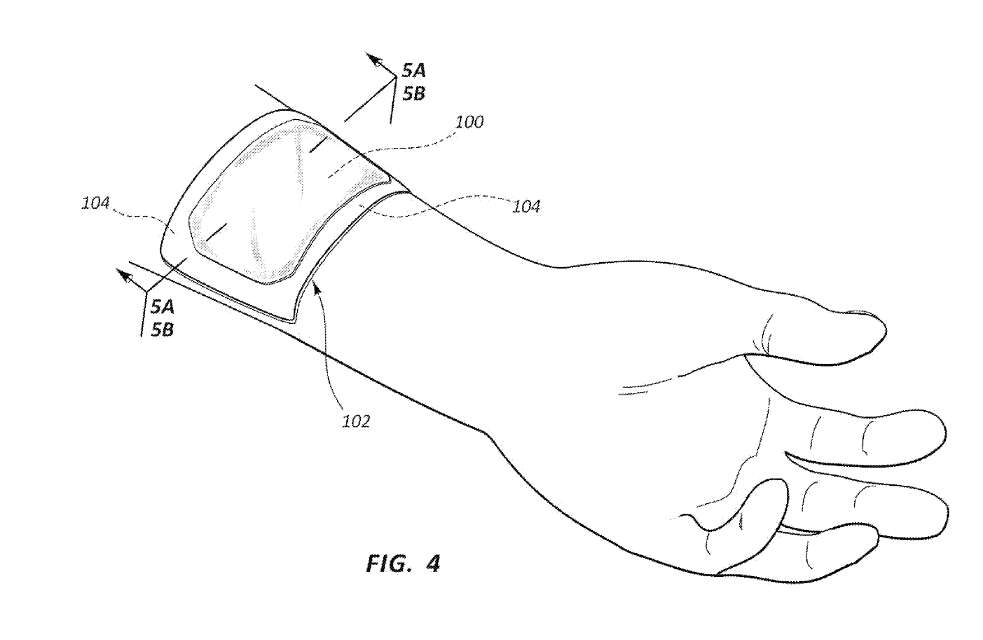

In one embodiment of the current disclosure, a method for delivering a drug across human skin is provided. Referring again to FIG. 2, this method comprises providing a structured sheet 102 comprising a drug retention area 103 and an adhesive elastic area 104 (the average MVTR of the drug retention area 103 is lower than 1,000 g/m²/24 hour, and can be lower than 100 g/m²/24 hour, the drug retention area 103 can be larger than about 50 cm², the average MVTR of the adhesive elastic area 104 can be higher than 1200 g/m²/24 hour, or even higher than 4,000 g/m²/24 hour), identifying a target human skin area, placing a liquid or semisolid formulation 110 comprising a drug onto the target human skin area or onto the drug retention area 103 of the structured sheet 102, placing the structured sheet 102 (with the formulation) onto the target skin area and maintaining it there for at least four hours. FIG. 4 is a perspective view of an embodiment of the current disclosure secured to a target skin area 100. As illustrated, the structured sheet 102 is kept on the target skin area 100 by the adhesion provided by the adhesive elastic area 104 of the structured sheet 102. The target skin area 100 does not contain an area with broken skin (i.e., broken stratum corneum layer, the skin's main barrier and outmost layer), because a compromised stratum corneum layer may allow much higher than pre-designed amounts of the drug to enter the body, which may be undesirable.

In another embodiment of the current disclosure, a method for treating joint pain or muscle pain is provided. This method comprises providing a structured sheet 102 comprising a drug retention area and an adhesive elastic area 104 (the average MVTR of the drug retention area is lower than 500 g/m²/24 hour, and the average MVTR of the adhesive elastic area 104 is higher than 4,000 g/m²/24 hour), placing a liquid or semisolid formulation comprising a local anesthetic or an anti-inflammatory drug onto a target skin area 100 under or adjacent to which a joint or muscular pain or a trigger point for musculoskeletal pain exists or onto the drug retention area of the structured sheet 102, placing the structured sheet 102 with the formulation on the target skin area 100, and maintaining it there for at least four hours. The structured sheet 102 is kept on the target skin area 100 by the adhesion provided by the adhesive elastic area 104 of the structured sheet 102. The drug is selected from the group consisting of lidocaine and/or other local anesthetic agents, and diclofenac and/or other nonsteroidal anti-inflammatory agents.

Joint and muscle pain include, but are not limited to, pain associated or caused by osteoarthritis, rheumatoid arthritis, joint or muscle injuries, and/or soft tissue injuries. A trigger point is a hyper-irritable point in a muscle that may radiate pain to broader areas. These areas may be distant from the trigger point itself.

FIG. 5A is a cross-section of the embodiment of FIG. 4 taken through line 5A-5A. In the illustrated embodiment, the drug formulation 110 is maintained in contact with the target skin area 100. The drug formulation 110 may remain at the drug retention area 103 for an extended period due at least in part to the barrier film 115 and the surrounding adhesive elastic area 104.

FIG. 5B is a cross-section of another embodiment. In the illustrated embodiment, the structured sheet 102 further comprises an absorbent layer 117 that covers all or part of the barrier film 115. The absorbent layer 117 may, in certain embodiments, be a fabric layer. In certain other embodiments, the absorbent layer 117 may be an open-cell sponge material. In operation, the drug formulation 110 is absorbed into the absorbent layer 117, thus allowing the formulation to contact the skin without flowing away

### EXAMPLES

To further illustrate these embodiments, the following examples are provided. These examples are not intended to limit the scope of the claimed invention, which should be determined solely on the basis of the attached claims.

### Example 1

### A Kit Comprising a Viscous Liquid Lidocaine Formulation

A kit of the current disclosure was prepared and tested on a human subject. The kit comprised two components: (1) a viscous lidocaine liquid formulation, and (2) a structured sheet with a release liner comprising a window.

The structured sheet and the release liner comprising a window were prepared by the following method. A 5 cm×12 cm portion was cut from a strip of an elastic and adhesive tape (KT TAPE® kinesiology therapeutic tape). The tape comprised an elastic fabric with an adhesive coating on one side. The tape could easily be stretched in one direction to increase its length by at least 10% (for example, increasing the original length of 12 cm to at least 13.2 cm), and the tape could easily recover its original length when the stretching force was removed. The tape was also breathable because the fabric was porous and the adhesive coating did not form a continuous layer. While the 5 cm×12 cm tape was stretched to about 13.2 cm, a thin polyethylene film with the dimension of about 3.6 cm×6 cm was placed onto the center of the adhesive side of the stretched tape. When the stretching force was removed, the tape recovered its original length of 12 cm and the polyethylene film's length shrank with the tape such that the polyethylene film was wrinkled and secured to the adhesive side of the tape. This tape with a polyethylene film at its center is one embodiment of the structured sheet in a kit of the current disclosure. The polyethylene film-covered area comprised the drug retention area of the structured sheet. The area of the 5 cm×12 cm tape that was not covered by the polyethylene film was the adhesive elastic area of the structured sheet.

Separately, a 3.5 cm×5 cm rectangular window was cut from the center of a piece of release liner (3M™ 9956 plastic release liner) with a dimension of 7 cm×15 cm. Thus, the 7 cm×15 cm release liner had a 3.5 cm×5 cm window in its center, wherein the 5 cm side of the window was in the same direction as the 15 cm side of the 7 cm×15 cm release liner. When the 5 cm×12 cm tape with the polyethylene film was placed onto the center of the release liner, wherein the adhesive side of the tape was in contact with the non-sticky side of the release liner, a 3.5 cm×5 cm area of the polyethylene film was not covered by the release liner but the rest of the structured sheet was covered by the release liner. The MVTR of the drug retention area of the structured sheet was estimated to be less than 100 g/m²/24 hour, mainly due to the polyethylene film. The MVTR of the adhesive elastic area was estimated to be higher than 5,000 g/m²/24 hour, as it was so porous that liquid water could permeate through it.

To generate the drug formulation, appropriate amounts of CARBOPOL® 981 NF polymer (a thickening agent) and NaOH (sodium hydroxide, used as a pH adjusting agent here) were added to distilled water to form, after sufficient stirring and waiting, a clear and viscous solution containing 1.6% CARBOPOL® 981 NF polymer and 0.9% NaOH. 2.5 parts of lidocaine base were added to 97.5 parts of the solution and well stirred to form a solid-in-water suspension, wherein the solid may be undissolved lidocaine, containing about 2.5% lidocaine base, 1.6% CARBOPOL® 981 NF polymer, and 0.9% NaOH. The suspension was heated to about 75° C. (above lidocaine base's melting temperature of about 68° C.) in an oven for about 30 minutes and stirred periodically during heating. The suspension was then allowed to cool to room temperature. Lidocaine particles stayed suspended in the suspension. This viscous suspension was the drug formulation component of the kit. The formulation was placed into a squeeze bottle comprising a long nozzle.

The formulation and structured sheet as described above were then tested on human skin. The above-described formulation was squeezed from the squeeze bottle onto the portion of the structured sheet's polyethylene film that was not covered by the release liner, the window area. The formulation was spread into a layer of about 1 mm thickness on the polyethylene film using the long tip of the squeeze bottle. The structured sheet with the formulation was then separated from the release liner and applied to the knee skin of a human subject. The adhesive elastic area of the structured sheet kept the structured sheet on the skin. The formulation on the skin was kept in a closed “pocket” because the drug retention area was surrounded by the adhesive elastic area in all directions. After about 90 minutes, the skin covered by the lidocaine formulation under the drug retention area was numb, as tested by scratching with a straightened paper clip. The structured sheet, along with the drug formulation, was removed after about six hours of application. At the time of the removal, the formulation on the skin was not dried, and the skin area was still numb. The human subject bent his knee in movements such as walking during the six-hour test period, and the structured sheet remained adhered to the skin.

In a separate test, the same lidocaine formulation was spread on an area of knee skin of the same human subject to form a 3.5 cm×5 cm formulation layer of about 1 mm thickness. The formulation layer was left on the skin for three hours and prevented from being touched by any external objects. The formulation layer dried after about 30 minutes, and the skin exposed to the formulation never numbed during the three-hour test period.

These results show that lidocaine can be delivered with the kit of the current disclosure into human skin at a high enough rate to numb intact human skin, and that such a delivery rate can be sustained for at least six hours. Without the structured sheet, the formulation cannot numb the skin because the vehicle solvent (i.e., water) evaporated shortly after the application started, and the transdermal absorption of lidocaine thus stopped, or was significantly slowed, because of the loss of the vehicle solvent. Vehicle solvents, such as water, are necessary for the permeation process.

Although the purpose of the formulation may not be to numb the skin, the ability to numb the skin and keep the skin numb for at least six hours indicates the presence of rapid and sustained transdermal delivery of lidocaine.

### Example 2

### A Kit Comprising a Less Viscous Lidocaine Formulation as Compared to Example 1

Another kit of the current disclosure for transdermal delivery of lidocaine was prepared and tested on a human subject. The kit comprised two components: (1) a viscous lidocaine liquid formulation (less viscous than the formulation of Example 1), and (2) a structured sheet with a release liner comprising a window.

To prepare the structured sheet and the release liner comprising a window, a 5 cm×12 cm portion was cut from a strip of an elastic and adhesive tape (KT TAPE® kinesiology therapeutic tape). Separately, a piece of barrier tape (3M™ 9830 tape) was laminated with a piece of an elastic non-woven fabric, using the tape's adhesive. A piece of the 3M™ 9830-non-woven laminate with a dimension of about 3.6 cm×about 6 cm was placed onto the center of the adhesive side of the 5 cm×12 cm tape. The 3M™ 9830 tape side of the laminate was in contact with the adhesive side of the elastic tape, such that the absorbent fabric was not covered. The structured sheet thus formed comprised the 5 cm×12 cm adhesive elastic tape with the 3.6 cm×5 cm of the laminate at its center, and the drug retention area of the sheet comprised the area with the fabric. The area of the 5 cm×12 cm tape that was not covered by the 3M™ 9830-non-woven fabric laminate was the adhesive elastic area of the structured sheet. Separately, the same release liner comprising a window at its center as that described in Example 1 was prepared. When the structured sheet was placed onto the center of the release liner (in a similar manner as in Example 1), a 3.5 cm×5 cm area of the 3M™ 9830-non-woven fabric laminate was not covered by the release liner but the rest of the structured sheet was.

The drug formulation in the current example comprised 0.4% CARBOPOL® 981 NF, 0.22% NaOH, and 2.5% lidocaine. Due to the lower concentration of CARBOPOL® 981 NF, the present drug formulation was less viscous than the formulation of Example 1. This formulation was the drug formulation component of the kit of the current disclosure.

With the release liner on the structured sheet, the above formulation was squeezed from the squeeze bottle through the window area in the release liner and onto the absorbent fabric of the drug retention area of the structured sheet. The amount of the formulation on the fabric was enough to saturate the fabric with the formulation (approximated 0.05 g to 0.1 g formulation per square centimeter of the fabric). The structured sheet with the formulation was then separated from the release liner and applied onto the wrist skin of a human subject. The adhesive elastic area of the structured sheet kept the structured sheet on the skin. The absorbent fabric of the drug retention area kept the formulation in contact with the skin and prevented it from flowing away from the drug retention area. After about 90 minutes, the skin covered by the lidocaine formulation under the drug retention area was numb. The structured sheet, along with the drug formulation, was removed after about six hours of application. At the time of the removal, the formulation absorbed in the fabric was not dried, and the skin area was still numb. The human subject used his wrist in normal household activities during the six-hour test period, and the structured sheet remained adhered to the skin.

In this example, the drug retention area comprised an absorbent fabric. The absorbent fabric acted to keep a slightly viscous drug formulation from flowing away from the drug retention area and kept the drug formulation in contact with the skin.

### Example 3

### A Kit Comprising a Diclofenac Solution

A kit for transdermal delivery of diclofenac for treating the pain associated with osteoarthritis of the knee comprises two components: a structured sheet and a diclofenac solution. The structured sheet in this example is similar to the structured sheet of Example 2, except the drug retention area has dimensions of 5 cm×20 cm and the entire structured sheet has dimensions of 7.5 cm×30 cm.

The drug formulation in this example is a liquid comprising about 1.5% diclofenac and about 45% dimethyl sulfoxide (a permeation enhancer), and other excipients. The drug formulation is similar to the PENNSAID® 1.5% diclofenac solution.

To use the kit, the user drips about 5 mL of the drug solution onto the drug retention area, and the solution is quickly absorbed into the absorbent fabric layer. The patient then applies the structured sheet on the knee so that the drug solution soaked drug retention area covers a horizontal rectangular skin area just under the patella. The adhesive and elastic area of the structured sheet adheres or secures the structured sheet, along with the drug solution, on the knee. In this way, the drug retention area keeps the solution in good contact with the skin, protects it from touching external objects, and significantly reduces the loss of volatile solvent(s) due to evaporation. The patient keeps the structured sheet with the formulation on the knee for 8 hours. Sufficient diclofenac is delivered during the application time so that the product is used only once a day instead of four times a day as required by the unprotected PENNSAID® solution.

### Example 4

### A Kit for Sustained Delivery of Lidocaine for at Least 12 Hours

A kit of the current disclosure was prepared and tested on a human subject. The kit comprised two components: (1) a viscous lidocaine liquid formulation, and (2) a structured sheet.

The structured sheet was prepared by the following method.

(1) A 5 cm×23 cm portion was cut from a strip of an elastic and adhesive tape (MUELLER Kinesiology Tape). The tape comprised an elastic fabric with an adhesive coating on one side. The tape could easily be stretched in one direction to increase its length by at least 10%, and could easily recover its original length when the stretching force was removed. The tape was also breathable because the fabric was porous and the adhesive coating did not form a continuous layer.

(2) A piece of non-woven absorbent fabric was laminated to a thin polyethylene film using a layer of transfer adhesive. A 3.5 cm×13 cm rectangle was cut from the laminate.

(3) The 3.5 cm×13 cm laminate from Step (2) was placed on the center of the adhesive side of the 5 cm×23 cm tape from Step (1), with the polyethylene film side of the laminate adhered to the adhesive side of the tape. The structured sheet was thus formed. The 3.5 cm×13 cm drug retention area, which had a layer of absorbent fabric and a barrier film on top of the tape, was surrounded by the adhesive elastic area (5 cm×23 cm except the center 3.5 cm×13 cm) in all directions.

(4) A 4% lidocaine formulation was made in the following way: appropriate amounts of CARBOPOL® 981 NF polymer (a thickening agent) and NaOH (sodium hydroxide) were added to distilled water to form, after sufficient stirring and waiting, a clear and viscous solution containing about 0.8% CARBOPOL® 981 NF polymer and 0.45% NaOH. 4 parts of lidocaine base (ground fine powder) were added to 96 parts of the solution and well-stirred to form a solid-in-water suspension, wherein the solid may be undissolved lidocaine. This viscous flowable liquid was the drug formulation component of the kit. The formulation was placed into a squeeze bottle comprising a long nozzle.

(5) The formulation and structured sheet as described above were then tested on human skin. The above-described formulation was squeezed from the squeeze bottle onto the fabric of the structured sheet's drug retention area and spread using the long tip of the squeeze bottle to form a layer of about 1.5-2 mm thickness covering almost the entire area of the drug retention area. The structured sheet with the formulation was then applied to the knee skin of a human subject, just below the patella, so the structured sheet formed a horizontal band across the knee that covered the front and most of the two sides of the knee. The adhesive elastic area of the structured sheet kept the structured sheet on the skin. The formulation on the skin was kept in a closed “pocket” because the drug retention area was surrounded by the adhesive elastic area in all directions. After about 12 hours, the structured sheet, along with the drug formulation, was removed. At the time of the removal, both the fabric and the skin surface were still wet, and the skin area was numb. The skin area was wiped clean with a damp KLEENEX® tissue, and the numbness continued for at least another hour. The human subject bent his knee in movements such as walking during the 12-hour test period, and the structured sheet remained adhered to the skin.

These results show that lidocaine can be delivered with the kit of the current disclosure into human skin at a high enough rate for a long enough time to keep intact human skin numb for a long sustained period of time. The 4% lidocaine formulation alone would qualify as an over-the-counter product in the U.S.A.

Again, although the purpose of the formulation may not be to numb the skin, the ability to numb the skin and keep the skin numb for at least 12 hours indicates the presence of rapid and sustained transdermal delivery of lidocaine.

### Example 5

### A Structured Sheet with a Drug Retention Area of 5 cm×15 cm

A structured sheet with a drug retention area of about 5 cm×15 cm and overall area of about 7.5 cm×25 cm was made. The component materials of the structured sheet were similar to that in Example 4. This larger structured sheet can be used to deliver lidocaine into the knee in a way similar to that in Example 4, or used to deliver diclofenac into the knee in a way similar to that in Example 3. The larger drug retention area and overall size of the structured sheet can allow more drug to be delivered.

The examples and embodiments disclosed herein are to be construed as merely illustrative and exemplary, and not a limitation of the scope of the present disclosure in any way. It will be apparent to those having skill in the art with the aid of the present disclosure that changes may be made to the details of the above-described embodiments without departing from the underlying principles of the disclosure herein. Moreover, the order of the steps or actions of the methods disclosed herein may be changed by those skilled in the art without departing from the scope of the present disclosure. In other words, unless a specific order of steps or actions is required for proper operation of the embodiment, the order or use of specific steps or actions may be modified. It is intended that the scope of the invention be defined by the claims appended hereto and their equivalents.

1. A kit for delivering a drug across human skin, comprising:
a liquid or semisolid formulation comprising a drug; and
a structured sheet comprising a drug retention area and an adhesive elastic area, wherein the drug is not for treating a wound or a skin infection, and wherein the moisture vapor transfer rate (MVTR) of the drug retention area is lower than one-fifth of the MVTR of the adhesive elastic area.

2. The kit of claim 1, wherein the MVTR of the drug retention area is lower than about 200 g/m²/24 hour.

3. The kit of claim 1, wherein the MVTR of the adhesive elastic area is higher than about 5000 g/m²/24 hour.

4. The kit of claim 1, wherein the MVTR of the drug retention area is lower than one-twentieth of the MVTR of the adhesive elastic area.

5. The kit of claim 1, wherein the drug retention area comprises a barrier film.

6. The kit of claim 1, wherein the drug retention area comprises a wrinkled barrier film.

7. The kit of claim 1, wherein the drug retention area is surrounded by the adhesive elastic area in all directions.

8. The kit of claim 1, further comprising a container, wherein the formulation and the structured sheet are contained in the container.

9. The kit of claim 1, further comprising a document comprising a use instruction which mentions the formulation and the structured sheet.

10. The kit of claim 1, further comprising a sheet of release liner comprising a window area, wherein the structured sheet and the release liner are positioned such that at least 80% of the adhesive elastic area of the sheet is covered by the release liner's non-window area, and at least a portion of the drug retention area coincides with the window area of the release liner such that the liquid or semisolid formulation can be applied to the drug retention area through the window area.

11. The kit of claim 1, wherein the drug retention area is elastic.

12. The kit of claim 1, wherein the drug is selected from at least one of lidocaine or another local anesthetic agent, diclofenac, ketoprofen, or another nonsteroidal anti-inflammatory agent.

13. The kit of claim 1, wherein the formulation comprises lidocaine at a concentration lower than about 5%.

14. The kit of claim 1, wherein the formulation comprises diclofenac at a concentration lower than about 3%.

15. The kit of claim 1, wherein the formulation is selected from at least one of an approved drug product or an over-the-counter product.

16. The kit of claim 1, wherein the formulation alone qualifies or would qualify as a generic version of an approved drug product in the U.S.A.

17. (canceled)

18. The kit of claim 1, wherein the formulation alone qualifies or would qualify as an over-the-counter product in the U.S.A.

19. A structured sheet for transdermal drug delivery, comprising:
a drug retention area; and
an adhesive elastic area, wherein the moisture vapor transfer rate (MVTR) of the drug retention area is lower than 1,000 g/m²/24 hour, wherein the drug retention area is larger than about 50 cm², and wherein the average MVTR of the adhesive elastic area is higher than 4,000 g/m²/24 hour.

20. A combination of the structured sheet of claim 19 and a sheet of a release liner comprising a window, wherein a side of the structured sheet is covered by the release liner such that at least 80% of the adhesive elastic area of the structured sheet is covered by the release liner's non-window area and at least a part of the drug retention area coincides with the window area of the release liner such that a portion of the drug retention area is not covered by the release liner.

21. (canceled)

22. A method for delivering a drug across human skin, comprising:
providing a structured sheet comprising a drug retention area and an adhesive elastic area, wherein the MVTR of the drug retention area is lower than 1,000 g/m²/24 hour, and wherein the average MVTR of the adhesive elastic area is higher than 4,000 g/m²/24 hour;
identifying a target human skin area, wherein the target human skin area does not contain an area comprising broken stratum corneum;
placing a liquid or semisolid formulation comprising a drug onto one of the target human skin area or the drug retention area of the structured sheet; and
placing the structured sheet onto the target human skin area, covering the drug formulation with its drug retention area, and maintaining it there for at least four hours, wherein the structured sheet is maintained on the target human skin area by the adhesion provided by the adhesive elastic area of the structured sheet.

23. A method for treating joint or muscle pain, comprising:
providing a structured sheet comprising a drug retention area and an adhesive elastic area, wherein the moisture vapor transfer rate (MVTR) of the drug retention area is lower than 1,000 g/m²/24 hour, and wherein the average MVTR of the adhesive elastic area is higher than 4,000 g/m²/24 hour;
placing a liquid or semisolid formulation comprising at least one drug, selected from the group consisting of a local anesthetic and an anti-inflammatory drug, onto a target human skin area, wherein the target human skin area is under or adjacent to at least one of a joint pain, a muscle pain, a trigger point for a muscle pain, or the drug retention area of the structured sheet;
placing the structured sheet and the formulation on the target human skin area; and
maintaining it on the target human skin area for at least four hours, and wherein the structured sheet is retained on the target human skin area by the adhesion provided by the adhesive elastic area of the structured sheet.

24. The method of claim 22, wherein the drug is at least one of lidocaine, another local anesthetic agent, diclofenac, or another nonsteroidal anti-inflammatory agent.
